(19) United States
(12) Patent Application Publication
(10) Pub. No.: US 2012/0196801 A1
(43) Pub. Date: Aug. 2, 2012

(54) Wound-healing, anti-ambustial, regenerating, and anti-viral pharmaceutical formula for local administration

(76) Inventors: Artur Martynov, Kharkov (UA); Boris S. Farber, Brooklyn, NY (US); Sonya Sophya Farber, New York, NY (US)

(21) Appl. No.: 12/931,457

(22) Filed: Feb. 1, 2011

(51) Int. Cl. A61K 31/472; A61P 17/02; A61P 31/12; A61K 38/28

(52) U.S. Cl. 514/6.5; 514/307

(57) ABSTRACT

The main active ingredients of the formula are papaverin, bendazol, and insulin. The drug may be produced in soft, liquid medicinal form or as an aerosol.

### TECHNICAL FIELD

This invention is related to pharmacology and cosmetology and may be used in the capacity of a medicinal and cosmetic substance for skin anti-aging, scar reduction, treatment of post-operative peritoneal commissures, increasing the speed of post-operative tissue regeneration, for the treatment of burns and increasing the speed of post-burn skin regeneration, and the treatment of viral dermatitis.

### PREVIOUS LEVEL OF TECHNOLOGY

This invention is related to pharmacology and medicine, specifically to pharmaceutical formulae for the treatment of illnesses caused by intracellular infectious agents, and may be used in the capacity of a substance for local application in dermatology, venerology, ophthalmology, and so on for the treatment of sores on skin and mucous membranes caused by herpes viruses, chlamidias, mycoplasms, and other intracellular infections, as well as being part of a set of preventive measures for the abovementioned illnesses. These diseases may be communicated through droplets in the air, sexual contact, transplantation, parenteral introduction, or daily contact. The pathological process may affect practically any organ in the human body, causing various nosological forms of the disease—skin, digestive organs, the urinary and sexual organs, the nerves, the cardiovascular system, respiratory system organs, visual organs, or the locomotive system—provoking oncological and auto-immune illnesses. The most widespread of the group of illnesses mentioned is viral diseases, in part, herpes viruses. According to WHO data, 95% of the world's population is infected with herpes viruses. Each year, in connection with the decline of the ecological situation in the world, our bodies' compensatory abilities, and the development of secondary immune deficits are noted in the increase of the number of people infected with viruses. Diseases caused by intracellular infectious agents are diseases of the entire body in their local forms. The tactic for the treatment of these diseases is the use at various stages of complex etiological and pathogenetic treatment directed at both the elimination of the reproduction of infectious agents and the increase of the body's immunological resistance. The problem of the treatment and prevention of these diseases is an important one, in part, to dermatology, venerology, and ophthalmology as a result of the clinically widespread manifestations of these diseases, which are seen in the skin and mucous membranes. For external, local application in the treatment and prevention of damaged skin and mucous membranes, medicinal substances are used of both synthetic and natural origin with various mechanisms of action: antioxidants, immune modulators, interferon generators, etc.

It is known that there is a drug for local application for the treatment of skin eruptions caused by herpes viruses: buthylhydroxytoluene (synonyms: dibunol, ionol) [1], whose active ingredient is 2,6-di-tert-butyl-4-metylphenol. The substance has a demonstrated antioxidant (antiradical) activity and is also used as an anti-tumor drug in the form of a cream liniment. A shortfall of the drug that is known may be considered to be that when it is applied locally, itching and skin reddening may occur. Substances of synthetic origin are also known: a drug for external use for prevention and treatment of sores and ulcers connected with the herpes virus, on the basis of carboxyl or dicarboxyl acid and their salts in combination with a base [2]; a drug containing pentanedial or hexanediol for local treatment of infectious diseases caused by viruses, bacteria, or fungi [3]; a drug for the treatment of herpes simplex that contains phosphate-1,4-imidazole-4-acetamine and a basis of water-soluble gel of vinyl polymer[4]. However, the drugs noted have a set of overall shortcomings: irritation may occur in the skin and mucous membranes of the patients; the drugs do not penetrate deeply into the body's tissues; they do not have immune-stimulating, wound-healing, regenerative, or anti-ambustial effects, and a long course of treatment is required with a high likelihood of relapses of the disease. In medical practice, certain drugs of plant origin are used that have some level of anti-viral activity. The following locally applied drugs are known [5]: alizarin from the Hedysarum alpinum L grass and H. flavescens and the bean family (Fabaceae), effective in relation to DNA-containing viruses of the herpes-group viruses (2% or 5% cream); helepin, the purified extract of the underground part of the Lespedeza hedysazoides (Pall.) Kitag and the bean family (Fabaceae) in the form of a 5% or 1% cream, which is used in herpes Zoster and herpes simplex, as well as on diseases of the mucous membranes of the mouth of viral origin; gossypol, a product obtained through the processing of the seeds or roots of the cotton plant (Gossypium sp.) of the Malvaceae family, which is active in relation to various viral strains, including dermatotropic strains of the herpes virus. The common shortfall of these drugs is their low pharmacological activity. It is known that when diseases caused by intracellular infectious agents disrupt the immune status, the viruses cause more severe illnesses with frequent and long-term relapses, which is connected with immune deficiency. On the other hand, the most persistent viral infection may cause slowing of the immune system and facilitate the development of secondary immune conditions. Thus a certain kind of “vicious cycle” arises, when on the background of an existing immune deficit, a virus acquires a recidivistic nature, and then, on its own, the virus maintains that condition through persistence and replication in immunocytes, in part in lymphocytes and macrophages, which are responsible for the creation of endogenous interferon. It has been proven that interferons (a group of low-molecular endogenous proteins with a molecular mass of from 15000 to 25000) have anti-viral, immune-modeling, and other biological properties and are one of the most important endogenous factors in the protection of the body against viral infection. The more interferon an organism produces, the better it is protected from viral infections. It is interesting that when interferons of natural or synthetic origin are used, a less immune-stimulating effect is observed than when a human body produces its own endogenous interferons. Clearly, it is of immediate interest to create drugs capable of stimulating the formation of endogenic interferons in the body.

### DISCLOSURE OF THE INVENTION

The goal of the invention is the creation of wound-healing, anti-ambustial, regenerative, and anti-viral pharmaceutical formulae for local application in which, through the use of papaverin and bendazol, an effect of mutual potentiating activity of the noted components is obtained and a demonstrated stimulation of formation of endogenous interferons is observed. As a result of the implementation of the invention, an effective substance with predominantly anti-viral and immunological, as well as (with the addition of insulin) reparative and anti-ambustial, activity for the treatment of diseases of the skin and mucus membranes caused by viruses, mycoplasms, chlamidias, and so on. As a prototype, a cream was chosen that contained 500 active units of interferon IVS [6]. This cream is obtained in the following manner: for 100 mg of cream, 500 active units of interferon (0.5% or 0.5 g) and 0.5 g of interferonogen IVS are made into a solution with 50 ml of distilled water and thickened with 49 g of polyethylene oxide with a mass of 400. Some of the shortcomings of the cream that is known include the fact that the introduction of interferon through the feedback effect slows the synthesis of endogenous interferon; there is no stimulating effect on the macrophages, lymphocytes or monocytes; the course of treatment in the hospital lasts for over a week; the cream is relatively expensive due to its content of bio-technical interferon. Also, this substance does not have regenerating or anti-ambustial properties.

The task set is achieved through the creation of a wound-healing, anti-ambustial, regenerating, and anti-viral pharmaceutical formula for local administration, which contains papaverin, bendazol, and, in certain cases, insulin, as its main active ingredients, and additionally contains pharmaceutically acceptable auxiliary morphogenic substances. Papaverin may be used as a base or in the form of an acidic salt, while bendazol may be used as a base or in the form of an acidic salt; in the capacity of insulin, human recombinant genetically engineered insulin, porcine insulin, or chemically modified insulin may be used. A wound-healing, anti-ambustial, re-generating, and anti-viral pharmaceutical formula is being prepared with an ingredient proportion of mass %: papaverin 0.001-3.0 mass %; bendazol 0.001-3.0 mass %; with the remainder being the base; in certain cases, insulin 0.001-3 mass % is added. This formula may be presented in a soft medicinal form (cream, liniment), a liquid medicinal form (water or non-water solution), or an aerosol or aerogel, and may contain a hydrophobic base, a hydrophilic base, or a diphilic base.

The active ingredients of the pharmaceutical formula being patented—papaverin, bendazol, and insulin—are known to be substances that affect the circulatory system and improve the metabolism of glucose [5]. Papaverin and bendazol are synthetic derivatives of isoquinoline and imidazole respectively and are myotropic spasmolytics that relax the smooth muscles of the blood vessels, bronchi, and other internal organs. Papaverin has a vessel-dilating, spasmolytic, and lightly sedative action and is traditionally used in medical practice as a spasmolytic against smooth muscle spasms in organs of the abdominal cavity, the bronchi, the urinary tract, the peripheric vessels, and the cerebral blood vessels. Bendazol has a myotropic, spasmolytic, vessel-dilating, and hypotensive action; it stimulates the functions of the spinal cord, and is used in practical medicine to treat smooth muscle spasms in blood vessels and internal organs, as well as in the treatment of nerve diseases. Bendazol has been used as a light-acting immune-stimulating drug. Separate information exists on the direct anti-viral activity in relation to influenza viruses for each of the substances presented. Bendazol is even included in the prevention scheme for influenza during the flu season. It is known that bendazol facilitates the strengthening of phagocytosis, leukopoesis, and antibody creation; that is, it also has immune-modeling activity and induces the synthesis of endogenous interferon. The interferonogenic properties of papaverin and insulin are not known from present sources of information. During the creation of the pharmaceutical formula being patented, the authors discovered for the first time the interferon-generating properties of these drugs. When papaverin, dibazol, and insulin are applied together in the pharmaceutical formula being patented, a synergetic effect is observed in the action of these drugs. The nature of this phenomenon is the following: bendazol is a selective activator of the synthesis of adenylate cyclase and facilitates a sharp increase in the speed of the synthesis of adenylate cyclase cAMP; papaverin is a selective inhibitor of PDEase, which damages cAMP; insulin, through the activation of cAMP, strengthens the accumulation of glucose in a cell. cAMP phosphorizes protein kinase, which, among other things, improves the synthesis of interferons. If bendazol is excluded from this scheme, there is not enough to synthesize cAMP, and accordingly, low titers of interferons will be observed that are insufficient to demonstrate anti-viral properties. Due to their general use, all three components activate for synthesis of interferons and stimulate cellular immunity (due to the stimulating activity of interferon gamma). Through experiments, the authors have established that the general use of papaverin, bendazol, and insulin condition the repeated potentiation of their direct anti-viral activity and mediated interferon-generating effect. In accordance with current practice in the therapy of cardiovascular system diseases, these components may be mixed in a single syringe for intramuscular or intravenous injections. Correspondingly, their general use in formulae for local application is completely safe. It has been established by research that a value interval of each of the active components of 0.001-3.0 mass % facilitates the necessary pharmacological activity and the technological indicators of the formula being patented. When the formula being patented contains less than 0.001 mass % of each of the active ingredients, it is not sufficiently active; increasing the content above 3.0 mass % is useless, because the pharmacological activity has nearly no increase after that, the price of the drug increases, and bendazol and papaverin cease to dissolve in the foundation and become a suspension. As a substance for local application, the formula being patented may be prepared in a soft form, primarily in the form of a cream (gel, liniment) or in liquid medicine form, primarily as a water solution. This formula, made in the form of a cream (gel, liniment), may contain any pharmaceutically acceptable foundation: hydrophilic, hydrophobic, or diphilic (mixed). The choice of foundations depends on the nature and localization of the illnesses diagnosed that were caused by intracellular infectious agents. The chosen foundation must facilitate the necessary rheologic indicators of the cream, be compatible with the active ingredients, answer the requirements of microbiological cleanliness, and provide the necessary shelf life. It is important but of no overall consequence that if necessary, a cream foundation with a limited shelf life may also contain a preservative. Standard, pharmaceutically acceptable substances can be used as preservatives. The authors studied formulae containing Nipagine and Decametoxin as preservative agents. In this case, the preservative agent content of the formula came to 0.04-4.0% mass %. Depending on the effectiveness of the preservative agents chosen, the content may vary. To obtain the pharmaceutical formula being patented in a soft medicinal form (for example, a cream), papaverin, bendazol and insulin are carefully ground and mixed with a foundation; the latter is added gradually while mixing until the creation of a homogeneous mass. The pharmaceutical formula being patented, when made in a liquid medicinal form (for example, a water solution) is prepared by dissolving the active ingredients, which had earlier been mixed in various mass portions, in, for example, water, until the creation of a true solution. The liquid medicinal form permits the use of the formula being patented as a skin wash, drops, or as urethral or vaginal instillations. The invention is illustrated by examples.

### BEST INVENTION IMPLEMENTATION OPTION

### Example 1

Determination of the effective interval for the qualitative values of active ingredients in the formula being patented, made in soft medicinal form, in part in the form of cream on a Vaseline-lanolin base, was conducted in experiments on chinchilla rats weighing 2.0-2.5 kg each. The criterion was the cream absorption ratio. On the sides of the experimental animals, a one-centimeter-square area was shaved, onto which 1 g of cream containing various concentrations of the active ingredients was applied. The control group of animals was given just the cream base without active ingredients. The areas were covered with polyethylene film, which was affixed with adhesive tape. In three hours, the film was removed and the skin was cleaned. With a biopsy needle, cell material was taken from the deep levels of subdermal tissue; the microphages therein were counted and the intensity of the fluorescence of the cytoplasms were determined. The substance's ability to penetrate into deep tissue layers and phagocyte itself with macrophages correlates with the level of fluorescence in the macrophages' cytoplasm. From the biopsy material, a drug was prepared and the level of cell fluorescence was measured. The absorption coefficient was determined to be the relationship of macrophage fluorescence in the control and the macrophage fluorescence after use of the cream being studied. The results of the experiment are presented in Table 1.

TABLE 1
Determination of the Effective Interval for Qualitative Values of
the Pharmaceutical Formula Being Patented, in the Form of a Cream

| | Content of Each of the Active | |
|---|---|---|
| Formula | Substances (mass %) (for | |
| Variations | equal substance balances) | Absorption Ratio, % |
| 1 | 0.001 | 2.50 ± 0.30 |
| 2 | 0.01 | 2.50 ± 0.30 |
| 3 | 0.1 | 3.00 ± 0.30 |
| 4 | 1 | 5.40 ± 0.50 |
| 5 | 1.5 | 4.70 ± 0.40 |
| 6 | 2.0 | 5.40 ± 0.50 |
| 7 | 2.5 | 5.40 ± 0.50 |
| 8 | 3.0 | 2.90 ± 0.30 |

The data in Table 1 indicate that various versions of the formulae with a content of each of the active ingredients from 0.001 to 3.0 mass % have a high absorption ratio. In this specific example, a rheologic indicator such as the possibility of making the versions being studied into cream on a Vaseline-lanolin foundation decreases somewhat when concentrations of the active ingredient are higher than 1.5 mass % in combination with the aforementioned type of foundation.

### Example 2

The reparative activity of the pharmaceutical formula being patented was studied in white rats with a mass of 100-120 g, which were given second-degree bums using a heated metal bar. The animals were divided into three groups of 15 rats each. The animals in the first group were treated with application to the bums of the substance being patented with an active ingredient content of 0.5% each every three days over the course of 30 days. Some of these animals received the substance being patented in the form of a cream with a Carbopol base, while the others received it in the form of a water solution. The animals in the second group were treated according to the previous scheme, but the prototype cream was used as a control. The third group was the control; the animals from this group were not treated. Every day, the scarring and epithelization of bums was registered until the day on which the signs of the burn disappeared. The regeneration ratio was considered to be the relationship between the healing period for the burn wound in treated animals as opposed to that in non-treated animals. According to the experimental data, the regeneration ratio for the pharmaceutical formula being patented in soft and liquid medicinal forms came to 36.8±2.2%; it was −1.2±0.1% for the prototype cream. Thus this formula exceeds the drug from the prototype by a factor of 35 in reparative activity.

### Example 3

The anti-viral activity of the formula being patented in relation to cytopathic viruses was studied in in vitro experiments in cultures of cells of animal origin, in part, cells from the kidneys of embryos of large horned stock. In the experiment, the formula being patented was used in the form of a cream with a content of 0.2 mass % of papaverin, dibazol, and insulin succinylated ant 3% on a hydrophilic base, Carbopol. The vesicular stomatitis virus, the coronavirus, and HSV-1 were used in the capacity of test viruses. In the experiment, 0.2 ml each of the corresponding virus in a working dosage (100 TCD50/0.2 ml) was introduced into a two-day cell culture, and 0.8 ml of supportive medium was added. In 24 hours, when a cytopathic effect appeared in the culture, the drug being patented was applied in various doses (with the exception of the control). The cells were incubated at 37° C. in a thermostat. A report on the experiment was conducted on the seventh day. A decrease in the titer of the virus under the influence of the drug being patented by 2 lg or more in comparison with the control was determined to indicate anti-viral activity. The data from the experiment are presented in Table 2.

TABLE 2
A Study of the Anti-Viral Activity of the Substance
Being Patented in Relation to Cytopathogenic Viruses

| | Minimum Effective | |
|---|---|---|
| | Concentration | Maximum Decline |
| | of the Substance Being | in Titer of the |
| Test Virus | Patented, mcg/ml | Virus lg TCD_{50/ml} |
| Vesicular | 1000 | 2.8 |
| Stomatitis Virus | | |
| Coronavirus | 2000 | 3.8 |
| HSV-1 | 2000 | 3.8 |

The data in Table 2 bear witness to the fact that the substance being patented indicates anti-viral activity against all viruses in the study. This permits us to draw the conclusion that the anti-viral activity of the substance being patented is not connected with specific peculiarities of the virus, but rather is based on mechanisms that are common to all cells. This mechanism seems to be the most reliable of all, in part, in the expressed interferon-generating activity of the drug being patented.

### Example 4

Immune modeling activity of the substance being patented was evaluated, in part, by its interferon-generating activity. The latter was tested upon non-linear white mice. The animals were infected intracerebrally with the vesicular stomatitis virus. The substance being patented and the comparison substances (Poly I:C and Pyrogenal) were introduced intraperitoneally in the form of water solutions of the creams (based on Carbopol) in a 0.9% solution of sodium chloride. On the first, third, fifth, seventh, and ninth days after introduction of the substances being tested, blood was taken from the animals for preparation of serum. The level of endogenous interferon induced by cells under the influence of the drugs being studied was determined in the blood serums in the cell culture-virus system with use of the micromethod on polystyrol 96-lunula planchettes. For a unit of activity, a reciprocal of end-point dilution of the serum was taken that caused 50% protection of the cells against the cytopathic activity of the test virus. The results of the experiment are presented in Table 3.

TABLE 3
Interferon-Generating Activity of the Substance Being Patented
in Comparison with Control Drugs Pyrogenal and Poly I:C

| | Minimum Dose Inducing | |
|---|---|---|
| Substance | Interferon, mcg/ml | Titer of ME Interferons |
| Substance Being | 1 | 1428 |
| Patented | | |
| Poly I:C | 5 | 1638 |
| Pyrogenal | 8.3 | 512 |

The data in Table 3 indicate that the interferon-generating activity of the drug being patented approaches that of the interferon inductor Poly I:C; however, at a minimum dose introducing interferon, it exceeds this means of comparison and definitely exceeds the interferon-generating activity of Pyrogenal. Additional study indicated that the substance being patented induces first and foremost the creation of beta and gamma interferons, and alpha interferons to a lesser degree. This fact may be explained by the therapeutic effect of the drug being patented in relation to various diseases caused by intracellular infectious agents in various models and in various cell systems. In the capacity of indicators of immune-modeling activity of the drug being patented are blast lymphocyte transformation reactions, the number of active T-lymphocytes, and phagocytosis. The values of these indicators, which were determined in in vitro experiments on the blood of guinea pigs, are presented in Table 4 for the substance being patented in comparison with the prototype.

TABLE 4
Immune Modeling Activity of the Substance Being Patented

| Immune Modeling | | Control, Drug from |
|---|---|---|
| Activity Indicators | Drug Being Patented, % | the Prototype, % |
| Blast Transfor- | 120 | 34 |
| mation Reaction | | |
| Active T-Lymphocytes | 72.0 ± 4.0 | 12.0 ± 1.0 |
| Phagocytosis | 32.0 ± 1.0 | 6.0 ± 0.5 |

According to the data in Table 4, the stimulation of lymphocytes in the blast lymphocyte reaction for the drug being patented exceeds that shown by the prototype by a factor of 4; correspondingly, the lymphocytes and the phagocytosis of the macrophages were more actively stimulated by a factor of 5. This indicates that the drug being patented does demonstrate immune-modeling activity.

### Example 5

A group of 46 volunteer patients was studied. Laboratory studies found that 10 of them had chlamidiosis, 14 had herpes, 15 had mycoplasmosis, and 7 had ureaplasmosis. The patients were divided into two groups of 23 each. While the base therapy was the same all over, the patients of one group were treated using the drug being patented in the form of a cream containing active ingredients at 0.2 mass % each on a base of Carbopol; the patients in the other group were treated with Cycloferon liniment. Both groups of patients were prescribed 0.5 g each of cream (liniment) daily until the clinical symptoms of the illnesses disappeared. The treatment results are presented in Table 6.

TABLE 5
Indicators of the Treatment of Illnesses Caused by Intracellular
Infectious Illnesses with the Use of the Substance Being
Patented in Comparison to Cycloferon Liniment

| | Patient | | Immune Modeling Effect | | |
|---|---|---|---|---|---|
| | Group, | Treat- | Blast | | |
| | Number | ment | Transfor- | Active | Phago- |
| | of | Period, | mation | T-Lympho- | cytosis, |
| Substance | People | Days | Reaction, % | cytes, % | % |
| Substance | 23 | 3-5 | 68 ± 2 | 73 ± 1 | 30 ± 0.2 |
| Being | | | | | |
| Patented, | | | | | |
| Cream | | | | | |
| Cycloferon, | 23 | 18-22 | 16 ± 1 | 15 ± 0.5 | 8 ± 0.2 |
| Liniment | | | | | |

As may be seen from the data shown in the table, the application of the formula being patented for the treatment of illnesses caused by intracellular infectious agents permits a reduction in the treatment time by a factor of 4 to 6 and to improve the body's resistance by a factor of 3 to 5 in comparison with the known substances. Thus the pharmaceutical formula being patented for local application is for the treatment of illnesses caused by intracellular infectious agents such as viruses, mycoplasms, chlamydia, and so on. The substance being patented has demonstrated reparative, anti-viral, and immune-modeling activity and shows an indirect interferon-generating effect; in stimulating the creation of endogenous interferon, it strengthens cellular and humoral immunity. The drug contains accessible components, is reasonably priced, and may be prepared in conditions provided by a standard pharmaceutical business. The variations in the medicinal form of the substance being patented facilitate an individual approach to patient treatment. The pharmaceutical formula being patented exceeds the therapeutic effectiveness of well-known analogous drugs and allows the treatment period to be significantly reduced.

### INDUSTRIAL APPLICABILITY

This invention is related to pharmacology and cosmetology and may be used in the capacity of a medicinal and cosmetic substance for skin anti-aging, scar reduction, treatment of post-operative peritoneal commissures, increasing the speed of post-operative tissue regeneration, for the treatment of burns and increasing the speed of post-burn skin regeneration, and the treatment of viral dermatitis. Unique equipment, high expenditures of energy, and original, difficult-to-access reagents are not necessary for the production of this formula, as the invention is industrially applicable on any pharmaceutical cream production equipment.

### REFERENCES

1. U.S. Pat. No. 5,215,748 A6135/78, 31/05. A drug for local application and a means of suppression of skin eruptions caused by the herpes virus.//Roy J/Mankovitz, Encino Calif.

2. Application No. 9602224 PCT (WO), A61K31/19. A drug for external application for the prevention and treatment of wounds and ulcers connected with the herpes virus./Sintov Amnom, Uran Rino; Agis Industries (1983) Ltd., Whalley Kevin.

3. Application No. 90/15597 PCM (WO) A61K31/045. A drug for local treatment of infectious diseases caused by viruses, bacteria, and fungi.

4. U.S. Pat. No. 5,294,440 A61K31/13. A drug for the treatment of herpes simplex./Bruce A. Jack, B. Thomas Wihte. Professional Inc.

5. Mashkovskiy M D. Medicinal drugs. A handbook for doctors. 14th ed. Moscow, OOO New Wave, Publisher: S. B. Divov, 2002, vol. 1, pp. 396-397, 398-399, 469. vol. 2, pp. 325, 335-336.

6. Kozlova V I, Maksumov S S, Pukhner A F. Viral diseases of the genitalia. Tashkent: Medicine, 1986.-247 pp.

1. A wound-healing, anti-ambustial, regenerating, and anti-viral pharmaceutical formula for local administration, including an effective quantity of active ingredients and pharmaceutically acceptable auxiliary morphogenic substances, distinct in that it contains papaverin and bendazol as its main active ingredients.

2. A wound-healing, anti-ambustial, regenerating, and anti-viral pharmaceutical formula for local administration according to claim 1, in which papaverin is presented as a base.

3. A wound-healing, anti-ambustial, regenerating, and anti-viral pharmaceutical formula for local administration according to claim 1, in which papaverin is presented in the form of an acidic salt.

4. A wound-healing, anti-ambustial, regenerating, and anti-viral pharmaceutical formula for local administration according to claim 1, in which bendazol is presented as a base.

5. A wound-healing, anti-ambustial, regenerating, and anti-viral pharmaceutical formula for local administration according to claim 1, in which bendazol is presented in the form of an acidic salt.

6. A wound-healing, anti-ambustial, regenerating, and anti-viral pharmaceutical formula for local administration according to any one of claims 1-5, distinct in that it is prepared with the following proportion of ingredients in mass %:
papaverin 0.001-3.0
bendazol 0.001-3.0 and the remainder is foundation.

7. A wound-healing, anti-ambustial, regenerating, and anti-viral pharmaceutical formula for local administration according to claim 6, distinct in that it is prepared in a soft medicinal form (cream, liniment).

8. A wound-healing, anti-ambustial, regenerating, and anti-viral pharmaceutical formula for local administration according to claim 6, distinct in that it is made in liquid (water or non-water solution) medicinal form.

9. A wound-healing, anti-ambustial, regenerating, and anti-viral pharmaceutical formula for local administration according to claim 6, distinct in that it is made into an aerosol or aerogel.

10. A wound-healing, anti-ambustial, regenerating, and anti-viral pharmaceutical formula for local administration according to claim 7, distinct in that it contains a hydrophobic base.

11. A wound-healing, anti-ambustial, regenerating, and anti-viral pharmaceutical formula for local administration according to claim 7, distinct in that it contains a hydrophilic base.

12. A wound-healing, anti-ambustial, regenerating, and anti-viral pharmaceutical formula for local administration according to claim 7, distinct in that it contains a diphilic base.

13. A wound-healing, anti-ambustial, regenerating, and anti-viral pharmaceutical formula for local administration, including an effective quantity of active ingredients and pharmaceutically acceptable auxiliary morphogenic substances, distinct in that it contains papaverin, bendazol, and insulin as its major active ingredients.

14. A wound-healing, anti-ambustial, regenerating, and anti-viral pharmaceutical formula for local administration according to claim 13, in which papaverin is presented as a base.

15. A wound-healing, anti-ambustial, regenerating, and anti-viral pharmaceutical formula for local administration according to claim 13, in which papaverin is presented in the form of an acidic salt.

16. A wound-healing, anti-ambustial, regenerating, and anti-viral pharmaceutical formula for local administration according to claim 13, in which bendazol is presented as a base.

17. A wound-healing, anti-ambustial, regenerating, and anti-viral pharmaceutical formula for local administration according to claim 13, in which bendazol is presented in the form of an acidic salt.

18. A wound-healing, anti-ambustial, regenerating, and anti-viral pharmaceutical formula for local administration according to claim 13, in which in the capacity of insulin, human recombinant genetically engineered insulin, porcine insulin, or chemically modified insulin may be used.

19. A wound-healing, anti-ambustial, regenerating, and anti-viral pharmaceutical formula for local administration according to any one of claims 14-18, distinct in that it is prepared with the following proportion of ingredients in mass %:
papaverin 0.001-3.0
bendazol 0.001-3.0
insulin 0.001-3.0
and the remainder is foundation.

20. A wound-healing, anti-ambustial, regenerating, and anti-viral pharmaceutical formula for local administration according to claim 19, distinct in that it is prepared in a soft medicinal form (cream, liniment).

21. A wound-healing, anti-ambustial, regenerating, and anti-viral pharmaceutical formula for local administration according to claim 19, distinct in that it is made in liquid (water or non-water solution) medicinal form.

22. A wound-healing, anti-ambustial, regenerating, and anti-viral pharmaceutical formula for local administration according to claim 19, distinct in that it is made into an aerosol or aerogel.

23. A wound-healing, anti-ambustial, regenerating, and anti-viral pharmaceutical formula for local administration according to claim 20, distinct in that it contains a hydrophobic base.

24. A wound-healing, anti-ambustial, regenerating, and anti-viral pharmaceutical formula for local administration according to claim 20, distinct in that it contains a hydrophilic base.

25. A wound-healing, anti-ambustial, regenerating, and anti-viral pharmaceutical formula for local administration according to claim 20, distinct in that it contains a diphilic base.
